(19) United States
(12) Patent Application Publication
(10) Pub. No.: US 2026/0248402 A1
(43) Pub. Date: Aug. 27, 2026

(54) PRESSURE-SENSING CATHETERS FOR IMPROVED FREQUENCY RESPONSE, AND RELATED DEVICES AND METHODS OF USE THEREOF

(71) Applicant: Laborie Medical Technologies Corp., Portsmouth, NH (US)

(72) Inventors: Jordan Peterson, South Jordan, UT (US); Jake Munger, Herriman, UT (US)

(21) Appl. No.: 19/549,797

(22) Filed: Feb. 25, 2026

Related U.S. Application Data
(60) Provisional application No. 63/762,843, filed on Feb. 25, 2025.

(51) Int. Cl. A61B 5/03; A61B 5/00; A61M 25/00

(52) U.S. Cl. CPC A61B 5/036; A61B 5/6853; A61B 2562/12; A61M 2025/0001; A61M 25/0009; A61M 25/0032; A61M 2025/004

(57) ABSTRACT

A pressure-sensing catheter includes an elongate catheter body having a proximal end, a distal end, and an outer surface. A primary lumen extends along at least a portion of the catheter body. One or more secondary lumens extend along at least a portion of the catheter body, and each secondary lumen has a non-circular cross-sectional shape. At least one collapsible vessel is on the outer surface of the catheter body. The collapsible vessel is fluidly connected to a secondary lumen of the one or more secondary lumens.

### CROSS-REFERENCE TO RELATED APPLICATIONS

This application claims the benefit under 35 U.S.C. § 119(e) of U.S. Provisional Patent Application Serial No. 63/762,843, filed February 25, 2025, the disclosure of which is hereby incorporated herein in its entirety by this reference.

### TECHNICAL FIELD

The present disclosure relates generally to pressure-sensing catheters and methods of using such catheters. In particular, embodiments of the present disclosure relate to a pressure-sensing catheter having secondary lumen(s) exhibiting a non-circular cross-sectional shape.

### BACKGROUND

Pressure catheter devices typically include an elongate pressure-sensing catheter having one or more gas-filled secondary lumens extending longitudinally through the catheter. An example of such a catheter is disclosed in U.S. Patent No. 11,350,838, which issued June 7, 2022, and is titled Pressure Catheter Device. A gas-filled (e.g., air-filled) membrane (commonly referred to as a balloon or a collapsible vessel) can be formed on an outer surface of a body of the catheter. The gas-filled membrane is in fluid connection with the one or more gas-filled secondary lumens. Changes in pressure against the gas-filled membrane result in changes in pressure of a fluid (e.g., air) within the one or more gas-filled secondary lumens. A pressure transducer connected to a proximal end of the one or more gas-filled pressure secondary lumens senses and displays or records the changes in pressure against the gas-filled membrane which is communicated through the one or more gas-filled secondary lumens to the pressure transducer.

Frequently, pressure-sensing catheters having a relatively small diameter (e.g., a French gauge 5 size catheter (e.g., a diameter of about 1.67 millimeters) or smaller) are used with pressure catheter devices. The body of such catheters exhibits a small cross-sectional area thus limiting the number and/or size of the gas-filled secondary lumens that can be incorporated in the pressure-sensing catheter. Sidewalls of the gas-filled secondary lumens are formed of a polymer material (e.g., an elastomer or thermoplastic) that forms the body of the catheter. As the size and/or the number of the gas-filled secondary lumens increases, a thickness of the polymer material surrounding the sidewalls of the gas-filled secondary lumens decreases, resulting in less amounts of the polymer material being exposed to a laser when welding the gas-filled membrane to the body of the catheter. This makes conventional pressure-sensing catheters sensitive to the energy of the laser to the degree that small deviations in the energy of the laser may result in excessive melting surrounding the sidewalls of the gas-filled secondary lumens. The excessive melting of the polymer material may result in occlusion of the gas-filled secondary lumens.

### BRIEF SUMMARY

According to one aspect of the disclosure, a pressure-sensing catheter has an elongate catheter body with a proximal end, a distal end, and an outer surface. A primary lumen extends along at least a portion of the catheter body. One or more secondary lumens also extend along at least a portion of the catheter body, and each secondary lumen has a non-circular cross-sectional shape. At least one collapsible vessel is on the outer surface of the catheter body, and the collapsible vessel is fluidly connected to a secondary lumen of the one or more secondary lumens.

According to another aspect of the disclosure, a pressure-sensing medical device includes a pressure-sensing catheter. The catheter has an elongate catheter body with a proximal end, a distal end, and an outer surface, and it includes a primary lumen extending along at least a portion of the catheter body. The catheter also includes one or more secondary lumens extending along at least a portion of the catheter body, and the one or more secondary lumens have a non-circular cross-sectional shape. At least one collapsible vessel is on the catheter body, and each collapsible vessel is fluidly connected to a secondary lumen of the one or more secondary lumens. The device also includes a pressure-sensing transducer positioned and configured to sense pressure within the at least one collapsible vessel and within the secondary lumen of the one or more secondary lumens.

According to another aspect of the disclosure, a method for manufacturing a pressure-sensing catheter is provided. The method includes extruding an elongate catheter shaft with a proximal end and a distal end, where a polymeric shaft wall defines a primary lumen extending between the proximal and distal ends and at least one secondary lumen extending between the proximal and distal ends. The at least one secondary lumen has a non-circular cross-sectional shape. In a distal portion of the catheter shaft, the method forms at least one opening between the at least one secondary lumen and an exterior of the catheter shaft. The method then positions a compliant member around the distal portion of the catheter shaft over the at least one opening, with a proximal cuff portion and a distal cuff portion that contact an outer surface of the catheter shaft. The compliant member is laser-welded to the catheter shaft to form a sealed chamber that is fluidly connected to the at least one secondary lumen, using four low-power laser passes that begin adjacent the first edge or the second edge of the compliant member and translate axially away to (i) conform the compliant member to the shaft and (ii) melt and fuse compliant member material to shaft material to create first and second weld seams. The first and second weld seams together seal the compliant member to the catheter shaft while keeping the primary lumen and the at least one secondary lumen open.

### BRIEF DESCRIPTION OF THE SEVERAL VIEWS OF THE DRAWINGS

For a detailed understanding of the disclosure, reference should be made to the following detailed description, taken in conjunction with the accompanying drawings, in which like elements have generally been designated with like numerals, and wherein:

FIG. 1 is a perspective view of a pressure-sensing catheter in accordance with an embodiment of the present disclosure;

FIG. 2 is an enlarged partial view of a distal end of the pressure-sensing catheter of FIG. 1;

FIG. 3 is a cross-sectional view of the pressure-sensing catheter of FIG. 1;

FIG. 4 is a cross-sectional view of another embodiment of the pressure-sensing catheter of FIG. 1 in accordance with the present disclosure;

FIG. 5 is a comparison between a cross-sectional area of the pressure-sensing catheter of FIG. 1 and a cross-sectional area of a previously known pressure-sensing catheter having circular secondary lumens;

FIG. 6 is a cross-sectional view of a pressure-sensing catheter with multiple collapsible vessels according to embodiments of the disclosure;

FIG. 7 is a cross-sectional view of a coaxial pressure-sensing catheter according to embodiments of the disclosure;

FIG. 8 illustrates a method for forming a pressure-sensing catheter according to embodiments of the disclosure;

FIG. 9 shows measured amplitudes of translated pressure signals of a body cavity measured using a conventional pressure-sensing catheter having secondary lumens with a circular cross-sectional shape; and

FIG. 10 shows measured amplitudes of translated pressure signals of a body cavity measured using a pressure-sensing catheter having secondary lumens with a non-circular cross-sectional shape in accordance with embodiments of the present disclosure.

### DETAILED DESCRIPTION

The illustrations presented herein are not actual views of any pressure-sensing catheter or any component thereof, but are merely idealized representations, which are employed to describe embodiments of the invention. The drawings are not necessarily to scale. Additionally, elements common between figures may retain the same numerical designation.

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, the term “substantially” in reference to a given parameter, property, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.). For example, “about” or “approximately” in reference to a numerical value may include additional numerical values within a range of from 90.0 percent to 108.0 percent of the numerical value, such as within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

As used herein, the terms “longitudinal” and/or “longitudinally,” used in reference to a pressure-sensing catheter or components thereof, are in reference to a major length of the pressure-sensing catheter. A “longitudinal” direction is a direction that is substantially parallel to the major length of the pressure-sensing catheter. The major length of the pressure-sensing catheter is defined by a length of the pressure-sensing catheter extending between a proximal end and a distal end of the pressure-sensing catheter and having a relatively large size compared to other dimensions (e.g., diameter) of the pressure-sensing catheter.

As used herein the term “distal,” when used in reference to a portion or region of a pressure-sensing catheter or a component thereof (e.g., a pressure-sensing catheter lumen), is used relative to the practitioner (not the patient), and means situated toward the end of the pressure-sensing catheter or secondary lumen that is inserted into the body of the patient during use.

As used herein the term “proximal,” when used in reference to a portion or region of a pressure-sensing catheter or a component thereof, is used relative to the practitioner (not the patient), and means situated toward the end of the catheter or lumen closest to the practitioner (and opposite the distal end) during use.

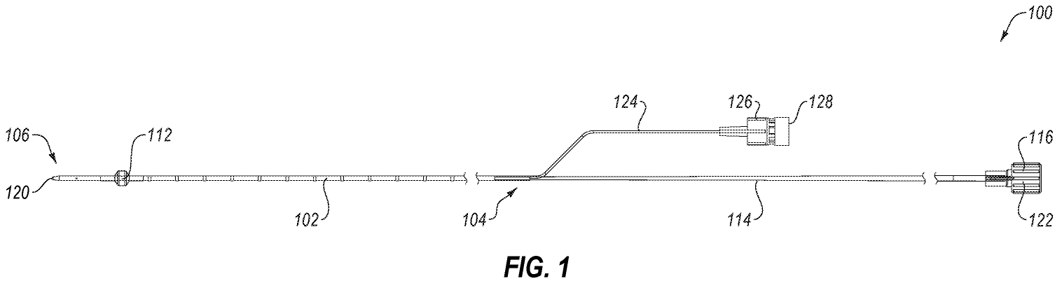

FIG. 1 illustrates a pressure-sensing catheter 100 in accordance with the present disclosure. The pressure-sensing catheter 100 may be used to measure the pressure within a body of a patient.

The structural parameters (e.g., the size, length, and volume of the pressure-sensing catheter of the present disclosure and components thereof), the operational parameters (e.g., working volume, non-working volume, operating pressure of the pressure-sensing catheter), and the material from which the pressure-sensing catheter of the present disclosure and components thereof are formed, may be as is disclosed in U.S. Patent No. 11,350,838, which issued June 7, 2022, and is titled Pressure Catheter Device, the disclosure of which is hereby incorporated herein in its entirety by this reference.

The catheter 100 has a catheter body 102, which may be at least substantially cylindrical in shape. The catheter body 102 may be formed from, for example, but not limited to, low-density polyethylene (LDPE) and/or high-density polyethylene (HDPE) material. The catheter body 102 extends from a proximal end 104 to a distal end 106. Referring briefly to FIG. 3, internal surfaces of the catheter body 102 define a primary lumen 108, and one or more secondary lumens 110.

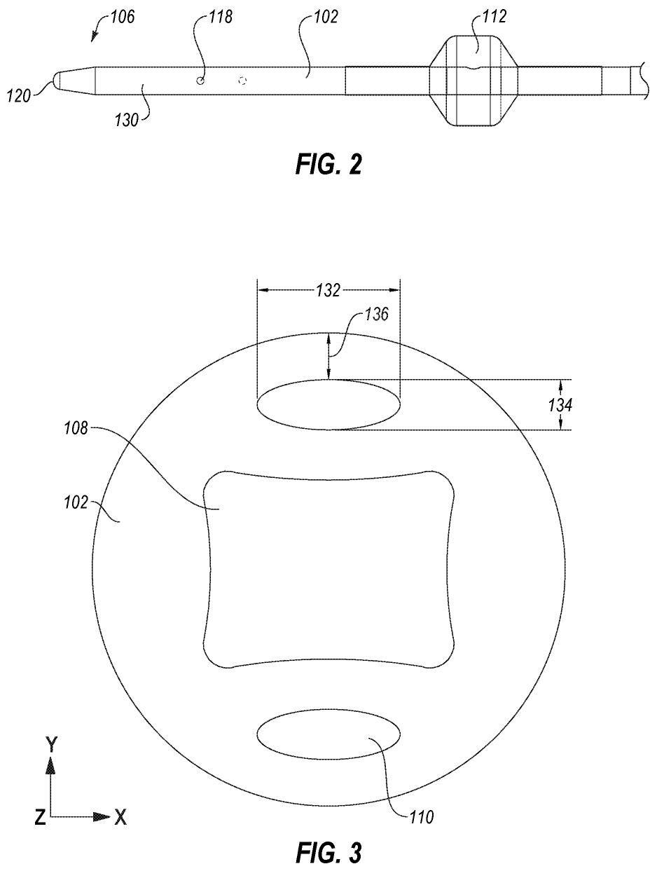

With continued reference to FIG. 1, the catheter 100 includes one or more compliant members in the form of collapsible vessels 112 (e.g., balloons) at the distal end 106 of the catheter body 102 of the catheter 100. FIG. 2 is an enlarged view of the distal end 106 of the catheter body 102. Each collapsible vessel 112 is in fluid communication with a respective secondary lumen 110 and is located and configured so as to at least partially (e.g., entirely) surround a relatively small section of the catheter body 102 of the catheter 100.

An elongate, tubular catheter extension 114 may extend from a proximal end 104 of the catheter body 102 to a proximal connector 116. The primary lumen 108 of the pressure-sensing catheter 100 is in fluid communication with a lumen of the tubular catheter extension 114. The proximal connector 116 in turn can be used to attach a syringe or other device (e.g., pump) used for the collection (e.g., aspiration) or delivery (e.g., infusion) of fluids to or from the cavity of the subject through holes 118 (FIG. 2) at the distal end 106 near a tip 120 of the catheter 100. The elongate tubular catheter extension 114 may be integrally formed with the catheter body 102 or attached thereto via an adhesive, or welding (e.g., laser welding). The proximal connector 116 may be or include a female luer lock, a male luer lock, or any other suitable connector. A cap 122 may be disposed on the proximal connector 116 prior to use.

Another elongate tubular catheter extension 124 that is in fluid communication with a secondary lumen 110 of the catheter may extend from the proximal end 104 of the catheter body 102 to a connector 126. The elongate tubular catheter extension 124 may be integrally formed with the catheter body 102 or attached thereto via an adhesive, or welding (e.g., laser welding). The connector 126 may include a female luer lock, a male luer lock, or any other suitable connection mechanism. A cap 128 may be disposed on the connector 126 prior to use.

The catheter 100 may include a separate tubular catheter extension 124 and connector 126 for each respective secondary lumen 110 and associated collapsible vessel 112 in fluid communication therewith.

The collapsible vessel 112 may be a flexible membrane (e.g., a balloon). The interior of each collapsible vessel 112 is in fluid communication with a corresponding secondary lumen 110 by way of an aperture extending through the catheter body 102 of the catheter 100. Fluid (e.g., air) may occupy an interior of the secondary lumen 110 and the collapsible vessel 112. The collapsible vessel 112 may be located and configured so as to entirely surround the catheter body 102. The collapsible vessel 112, which is filled with gas (e.g., air), is configured to deflect or deform upon application of pressure thereto, and to expand again upon removal of the pressure therefrom. The collapsible vessel 112 may be secured to the catheter body 102 by laser welding, adhesive bonding, RF welding, induction welding, hot air welding, or other suitable methods known in the art.

As discussed in further detail below, embodiments of the present disclosure are particularly relevant to small diameter catheters such as size French gauge 5 or smaller. For example, a diameter of the catheter body 102 may be about 2 millimeters or smaller, such as between about 1 millimeter and about 2 millimeters. In some embodiments, the diameter of the body may be about 1 millimeter (e.g., French gauge 3), about 1.33 millimeters (e.g., French gauge 4), or about 1.67 millimeters (e.g., French gauge 5). However, embodiments of the present disclosure may be applied to other sized catheters such as French gauge 7 catheters. Furthermore, embodiments of the present disclosure may be applied to coaxial type catheters.

When an outer diameter of the catheter is constrained—such as in small French-size catheters—improving pneumatic frequency response by simply increasing an inflation/sensing lumen diameter is often impractical. Increasing lumen diameter within a fixed outer diameter generally requires reducing wall thickness, which can compromise tensile strength, kink resistance, and burst resistance. Furthermore, increasing lumen diameter within a fixed outer diameter may compromise overall manufacturability and may also violate minimum wall requirements needed for reliable welding or bonding of distal components (e.g., a balloon), which can lead to occlusion of the secondary lumen. Conversely, maintaining adequate wall thickness can limit achievable lumen cross-sectional area, increasing flow resistance and thereby degrading pressure transmission bandwidth.

In such outer diameter limited designs, optimizing the shape of the secondary lumen (rather than its nominal diameter) may provide increased pneumatic frequency response while maintaining appropriate wall thickness. For example, by adopting a non-circular cross-sectional geometry in a secondary lumen that more efficiently occupies available cross-sectional “real estate” while preserving required polymer thickness, the catheter can increase effective lumen area and reduce flow resistance without increasing an outer diameter of the catheter or materially compromising structural integrity.

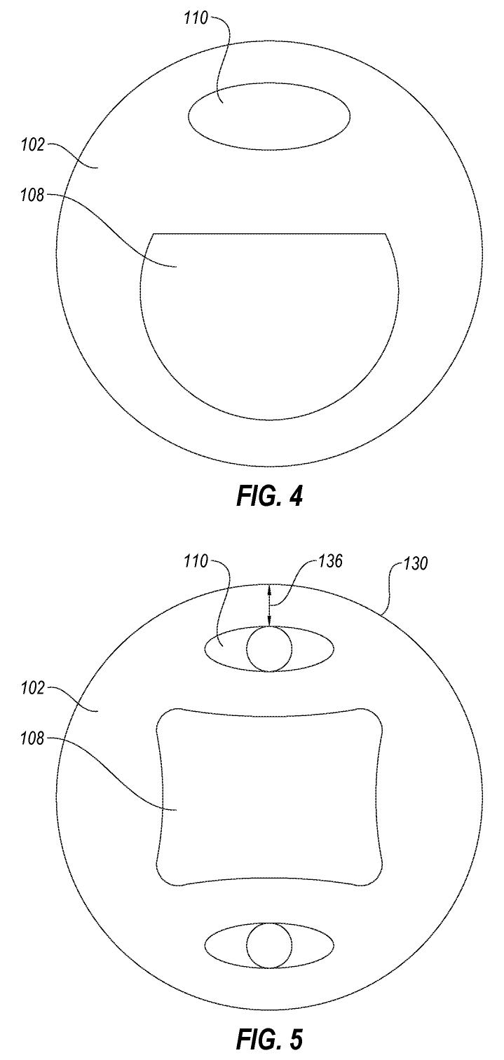

With reference to FIGS. 3-4, the secondary lumens 110 of the catheter 100 are formed to have a non-circular cross-section. For example, the secondary lumens 110 may have an elliptical cross-sectional shape as shown in FIGS. 3-4. However, other non-circular cross-sectional shapes may also be used for the secondary lumens 110 such as an oval cross-sectional shape or a “D” cross-sectional shape. In embodiments wherein the secondary lumen 110 exhibits an elliptical shape, a length 132 along a major axis (e.g., in the X direction in FIG. 3) of a cross-section of the secondary lumen may be in a range extending between about 0.20 millimeters and about 0.55 millimeters for a French gauge 5 catheter, and a width 134 along a minor axis (e.g., in the Y-direction in FIG. 3) of a cross-section of the secondary lumen may be in a range extending between about 0.15 millimeters and about 0.25 millimeters for a French gauge 5 catheter. In some embodiments, a ratio of the major axis length to the minor axis length of the cross-sectional area of the secondary lumen 110 may be from about 3.7:1 or smaller.

For a French gauge 5 catheter, a minimum distance (e.g., a wall thickness) between the secondary lumen 110 and an outer surface 130 of the catheter body 102 may be in a range between about 0.15 millimeters and about 0.25 millimeters. Similarly, a minimum distance between the secondary lumen 110 and the primary lumen 108 may be in a range between about 0.15 millimeters and about 0.25 millimeters.

The primary lumen 108 may have a rectangular shape with inwardly arcing (e.g., concave) sides as shown in FIG. 3, a “D” shape as shown in FIG. 4, a rectangular shape with straight edges, a circular shape, or other predetermined shapes. In some embodiments, the pressure-sensing catheter 100 includes two secondary lumens 110 located on opposite sides of the primary lumen 108, as shown in FIG. 3. The two secondary lumens 110 may exhibit a similar cross-sectional size and shape. In some embodiments, more or less than two secondary lumens 110 may be incorporated into the catheter 100, such as four secondary lumens 110 or one secondary lumen 110.

For a French gauge 5 catheter, the one or more secondary lumens 110 may have a cross-sectional area of between about 0.020 square millimeters and about 0.095 square millimeters, such as about 0.050 square millimeters or less, or even about 0.025 square millimeters or less. The cross-sectional area of the primary lumen 108 may be between about 0.35 square millimeters and about 0.80 square millimeters. Furthermore, the lumens 108 and 110 may be sized such that the ratio of a total cross-sectional area of the lumens 108 and 110 to a total cross-sectional area of the pressure-sensing catheter 100 is at least about 0.25, or even at least about 0.27.

In some embodiments, the primary lumen 108 may have a rectangular cross-sectional shape with straight sides and the one or more secondary lumens 110 may have an oval or elliptical cross-sectional shape. In additional embodiments, the primary lumen 108 may have a rectangular cross-sectional shape (e.g., square) with concave or straight sides and the one or more secondary lumens 110 may have a “D” cross-sectional shape with sharp or rounded corners. Amplitudes of pressure signals measured using such embodiments may have a greater value than an amplitude of pressure signals measured using embodiments wherein the primary lumen 108 exhibits a rectangular shape with straight sides and the one or more secondary lumens 110 exhibit an oval or elliptical shape.

During use, a liquid source such as a liquid-filled syringe or a pump, for example, may be coupled to the primary lumen 108 by way of the proximal connector 116 and configured to enable flow of the liquid through the primary lumen 108. The connector 126 is coupled to a sensing module to form a medical device comprising the catheter 100 and the sensing module. The sensing module includes at least one pressure-sensing transducer located and configured to sense a pressure within the at least one collapsible vessel 112 and the secondary lumens 110 of the catheter 100. The sensing module may also include a microprocessor and memory and other circuitry for sensing, monitoring, and/or recording an electrical signal generated by the pressure-sensing transducer relating to the sensed pressure or pressure fluctuations. The sensing module may be configured to generate and output data relating to the sensed pressure within the body of the patient to the practitioner using the device relating to the pressure, which data can then be used by the practitioner for diagnosis and/or treatment.

FIG. 5 is a schematic cross-sectional diagram superimposing a conventional pressure-sensing catheter with secondary lumens having a circular cross-sectional shape onto a pressure-sensing catheter 100 having secondary lumens with a non-circular shape as described herein. As can be seen in FIG. 5, the secondary lumens 110 have a larger cross-sectional area relative to the circular lumens while maintaining the same wall thickness 136 between the secondary lumens 110 and an outer surface 130 of the catheter body 102. Compared with the circular secondary lumens, the non-circular secondary lumens 110 of the pressure-sensing catheter 100 of the present disclosure provide a greater cross-sectional area for translating changes in pressure to pressure signals down the secondary lumens 110, while maintaining an acceptable wall thickness 136 of polymer material between the secondary lumens 110 and the outer surface 130 of the catheter body 102 and between the secondary lumens 110 and the primary lumen 108. Accordingly, the pneumatic frequency response of the catheter 100 may be increased without increasing the chance of occlusion of the secondary lumens 110 during manufacturing of the catheter 100.

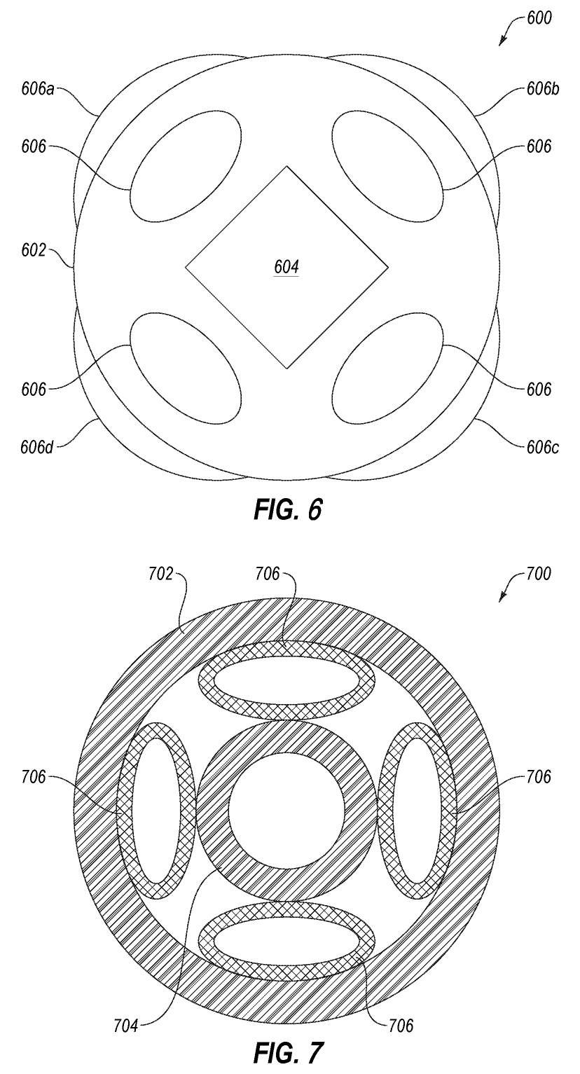

With reference to FIG. 6, in some embodiments the present disclosure includes a pressure-sensing catheter 600 configured to provide pressure sensing at a plurality of circumferential locations such as at a common axial position along the catheter. The catheter 600 includes a catheter body 602 defining a primary lumen 604 and a plurality of secondary lumens 606 extending longitudinally through at least a portion of the catheter body 602. The plurality of secondary lumens 606 are configured with a non-circular cross-sectional area. A plurality of compliant members in the form of collapsible vessels 606a-606d (e.g., balloons) are disposed on the catheter body 602 at, for example, substantially the same longitudinal distance from a distal end (e.g., the distal end 106 in FIG. 1) of the catheter 600, while being positioned at different circumferential locations around the catheter body 602. Each collapsible vessel 606a-606d is in fluid communication with a respective secondary lumen 606 such that pressure applied at a particular circumferential position (e.g., relative to a selected one of the collapsible vessels 606a-606d) produces a corresponding pressure change within the associated secondary lumen 606, thereby enabling circumferentially resolved pressure sensing.

In these embodiments, the collapsible vessels 606a-606d may be secured to the catheter body 602 by any suitable technique described herein (e.g., welding or bonding) and may be configured to deform in response to external pressure to pneumatically transmit a pressure signal through the corresponding secondary lumen 606 to proximal sensing hardware. Accordingly, the configuration shown in FIG. 6 can facilitate detection of pressure differentials as a function of circumferential position at a given axial location (e.g., radial or circumferential pressure variations), while maintaining the packaging benefits of providing the secondary lumens 606 within the catheter body 602.

Although FIG. 6 illustrates collapsible vessels 606a-606d positioned at substantially the same axial distance from the distal end of the catheter 600, in other embodiments one or more collapsible vessels may additionally or alternatively be positioned at different axial distances along a length of the catheter body 602. For example, the catheter 600 may include collapsible vessels located at multiple longitudinal positions to sense pressure at multiple axial locations, with each collapsible vessel being in fluid communication with a respective secondary lumen 606 (or with selected secondary lumens being coupled to sensing hardware in a multiplexed arrangement).

With reference to FIG. 7, in some embodiments the present disclosure includes a coaxial catheter 700 in which multiple pressure-sensing flow paths are provided using a tube-in-tube architecture. The coaxial catheter 700 includes an outer catheter 702 that defines an outer boundary of the coaxial catheter 700. A primary catheter 704 is disposed within the outer catheter 702, for example generally concentrically as shown, and may define a primary passage for one or more functions such as fluid delivery, fluid drainage, guidewire passage, or other catheter functions. A plurality of secondary catheters 706 are also disposed within the outer catheter 702, radially between the outer catheter 702 and the primary catheter 704 and arranged at different circumferential positions around the primary catheter 704.

In these embodiments, each secondary catheter 706 can define a respective secondary passage configured to pneumatically transmit a pressure signal (e.g., from a distal pressure interface such as a collapsible vessel) toward a proximal sensing interface. The secondary catheters 706 may be distributed substantially symmetrically around the primary catheter 704, or in any other circumferential pattern suitable for the intended sensing locations and packaging constraints. Further, one or more of the secondary catheters 706 may have a non-circular cross-sectional profile (e.g., generally flattened, elliptical, D-shaped, or otherwise non-circular) to increase flow area and/or reduce pneumatic resistance while fitting within the limited annular space between the primary catheter 704 and the outer catheter 702. By using the coaxial arrangement of FIG. 7, the overall outer diameter of the coaxial catheter 700 can be maintained while increasing the aggregate cross-sectional area available for pressure-sensing passages relative to arrangements in which all passages must be formed within a single monolithic catheter wall.

The coaxial catheter 700 may be manufactured by extruding the outer catheter 702, the primary catheter 704, and the secondary catheters 706 separately, followed by assembling the primary catheter 704 and secondary catheters 706 within the outer catheter 702. In some embodiments, the assembled components may be secured relative to one another by bonding, thermal fusing, reflow, or other joining techniques such that the secondary catheters 706 remain at desired circumferential positions along at least a distal sensing region of the coaxial catheter 700. In other embodiments, one or more of the components may be co-formed or over-formed (e.g., by over-extrusion or lamination) to capture and retain the relative positions of the primary catheter 704 and the secondary catheters 706 within the outer catheter 702. The secondary catheters 706 may extend along all or a portion of the length of the coaxial catheter 700, and may be individually coupled to distinct proximal sensing channels to enable independent sensing, and/or selectively coupled to a common sensing channel to enable multiplexed sensing.

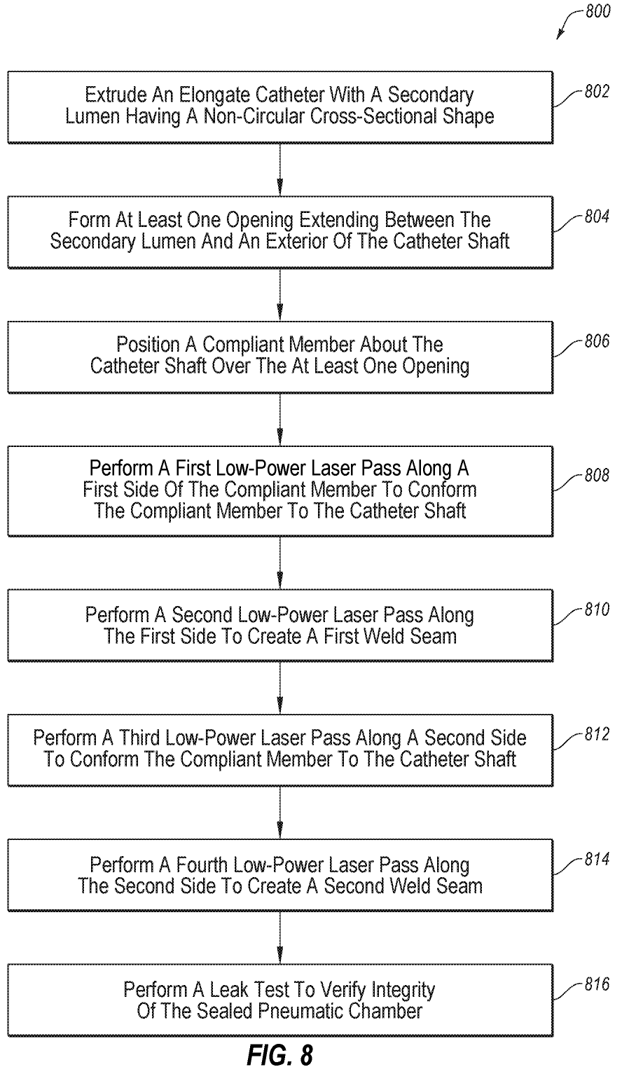

With reference to FIG. 8, a method 800 for manufacturing a pressure-sensing catheter (e.g., catheter 100 and/or catheter 600) is described. Although the acts of FIG. 8 are shown and described in a particular order, in other embodiments certain acts may be performed in a different order, repeated, omitted, and/or combined, and the method 800 may be used to manufacture any of the catheters described herein.

In act 802, the method 800 includes extruding an elongate catheter shaft (e.g., catheter body 102 or catheter body 602) having a proximal end and a distal end, the catheter shaft including a polymeric wall defining a primary lumen (e.g., primary lumen 108 or primary lumen 604) and at least one secondary lumen (e.g., secondary lumen 110 or secondary lumen 606). In some embodiments, the secondary lumen(s) are extruded to have a non-circular cross-sectional shape (e.g., generally flattened, elliptical, D-shaped, or otherwise non-circular) selected to increase cross-sectional flow area and improve pneumatic pressure transmission while maintaining minimum wall thickness required for structural integrity and manufacturability.

In some embodiments, the extrusion tooling (e.g., die and/or mandrel geometry) is configured to account for polymer flow and post-extrusion relaxation/shrinkage such that the as-cooled secondary lumen exhibits a desired non-circular profile. For example, the tooling may “over-form” the primary lumen profile and/or the secondary lumen profile (relative to a target final shape) to compensate for dimensional changes during cooling, draw-down, and/or subsequent thermal processing, thereby maintaining the desired lumen geometry.

In act 804, the method 800 includes forming, in a distal portion of the catheter shaft, at least one opening that fluidly couples the at least one secondary lumen to an exterior surface of the catheter shaft. The opening may include one or more holes 118 formed through the catheter wall (e.g., by mechanical piercing, laser drilling, punching, skiving, or other suitable techniques) at a location corresponding to a distal sensing region. In some embodiments, a plurality of holes 118 are formed in a circumferential and/or axial pattern to promote fluid communication between the secondary lumen and an overlying compliant member, while preserving wall strength and minimizing disruption to the primary lumen.

In act 806, the method 800 includes positioning a compliant member about the distal portion of the catheter shaft over the opening(s). In some embodiments, the compliant member includes a collapsible vessel (e.g., collapsible vessel 112 or, in multi-vessel embodiments, collapsible vessels 606a-606d) that overlies the opening(s) and is configured to deform in response to external pressure to transmit pressure pneumatically through the associated secondary lumen. The compliant member can include a proximal cuff portion and a distal cuff portion contacting an outer surface 130 of the catheter shaft to define a region intended to become a sealed chamber upon attachment. In some embodiments, prior to welding/bonding, the catheter shaft and/or the compliant member may be cleaned and/or temporarily lubricated to facilitate assembly; for example, the compliant member may be placed using an alcohol bath or alcohol wetting to reduce handling friction and assist positioning, after which the alcohol is removed/evaporated during subsequent processing.

In act 808, act 810, act 812, and act 814, the method 800 includes laser-welding the compliant member to the catheter shaft to form a sealed chamber in fluid communication with the at least one secondary lumen. In some embodiments—such as for small outer diameter catheters where wall thickness is limited—the compliant member is welded using two low-power laser passes on each of two opposed sides, with each pass beginning adjacent an edge of the compliant member and translating axially away from the compliant member. As used herein, a “low-power” laser pass refers to a welding scan performed with the laser commanded at a duty-cycle (PWM) or power setting that is a small fraction of the laser’s rated maximum output power (e.g., no more than about 10% of the rated maximum). As one non-limiting example, when a 30 W RF-excited Synrad® CO₂ laser is controlled using a Synrad® UC-2000 controller that commands output via PWM duty-cycle percentage, a 4% setting corresponds to a commanded duty cycle of about 4% and an average optical output on the order of about 1.2W, recognizing that laser output is approximately proportional to PWM duty cycle and may vary with operating conditions and modulation frequency. In some embodiments, a “low-power” laser pass may be an average optical output of about 3W or less. In some embodiments, a “low-power” laser pass may be an average optical output of about 1.5W or less.

For example, in act 808, the method 800 performs a first low-power laser pass along a first side of the compliant member such as along one of the proximal or distal cuff portions of the compliant member. In some embodiments, this first pass is a conditioning/shaping pass performed at an energy level selected to soften and conform the compliant member to the catheter shaft without materially collapsing or occluding the lumens, and (when alcohol wetting is used) to drive off residual alcohol at the weld interface. In addition, in embodiments where the cuff portion is heat-shrinkable or otherwise thermally responsive, the first pass may cause the cuff to shrink or draw into intimate contact with the outer surface 130 of the catheter shaft, thereby improving interface contact and weld consistency.

In act 810, the method 800 performs a second low-power laser pass along the same first side, again beginning adjacent the compliant member and translating axially away. In some embodiments, the second pass is performed at an energy level and/or with a dwell time sufficient to melt and fuse the compliant member material to the catheter shaft material, thereby forming a first weld seam.

After completing both passes on the first side, the laser process is repeated on the opposite side of the compliant member. In act 812, the method 800 performs a third low-power laser pass along a second side of the compliant member opposite the first side, the third pass beginning adjacent the compliant member and translating axially away. Act 812 may be similar to act 808 to soften and conform the compliant member to the catheter shaft, to drive off residual alcohol at the weld interface (when alcohol wetting is used), and/or to cause the cuff to shrink or draw into intimate contact with the outer surface 130 of the catheter shaft.

In act 814, the method 800 performs a fourth low-power laser pass along the second side, again beginning adjacent the compliant member and translating axially away, to melt and fuse the materials and form a second weld seam. In this manner, the first and second weld seams cooperate to seal the compliant member to the catheter shaft and define a sealed pneumatic chamber over the opening(s), while maintaining patency of the primary lumen (e.g., primary lumen 108/604) and the at least one secondary lumen (e.g., secondary lumen 110/606). In some embodiments, the “low power” passes are implemented as multiple overlapping scans, and parameters such as scan speed, spot size, overlap, number of passes, and/or focal position are selected to reduce peak heat input and thereby mitigate lumen deformation, wall thinning, or occlusion. In other embodiments, additional low-power passes may be used (e.g., more than two per side) to further distribute heat input while still achieving a hermetic or near-hermetic seal.

In some embodiments, the welding acts (acts 808-814) may be performed in a different order. For example, acts 808 and 812 may be performed to provide a low-power laser bass along both sides of the compliant member. Then, acts 810 and 814 may be performed as a final low-power laser pass along both sides of the compliant member. In some embodiments, each welding pass may begin at a position away from the compliant member and may translate axially towards the compliant member.

In some embodiments, to further reduce risk of lumen deformation and/or occlusion during the welding acts (acts 808-814), the method 800 optionally includes inserting a temporary support member (e.g., a wire, mandrel, or stylet) into one or more lumens before welding and removing the support member after welding. For example, a mandrel may be inserted into the at least one secondary lumen (e.g., secondary lumen 110/606) and/or the primary lumen (e.g., primary lumen 108/604) to maintain lumen geometry and/or to act as a heat sink during laser processing. The temporary support member may comprise any suitable material (e.g., stainless steel, nitinol, or other metal) and may be sized to support the lumen without causing permanent deformation; in some embodiments, the support member is coated or otherwise treated to facilitate removal.

In act 816, the method 800 includes performing a leak test to verify integrity of the sealed chamber and associated fluidic pathway. In some embodiments, leak testing includes pressurizing the sealed chamber through the associated secondary lumen and monitoring pressure decay over time, monitoring flow required to maintain a target pressure, and/or performing a bubble test (e.g., submerging at least the distal portion and observing for bubbles) to confirm that the weld seams provide an adequate seal. In embodiments including multiple compliant members (e.g., collapsible vessels 606a-606d), the leak test may be performed independently for each associated secondary lumen pathway, thereby enabling verification of sealing and channel isolation for each sensing channel.

In the embodiments described herein, the fluid contained within and/or communicated through the secondary lumen (e.g., secondary lumen 110 or secondary lumen 606) and any associated collapsible vessel (e.g., collapsible vessel 112 or collapsible vessels 606a-606d) may include air. However, other working fluids may additionally or alternatively be used. For example, in some embodiments the working fluid comprises another compressible gas, such as nitrogen, carbon dioxide, or a noble gas, selected based on availability, sterility, compatibility with catheter materials, and desired pressure-transmission characteristics. While liquids (e.g., saline or water) may also be used as a working fluid in certain implementations, the advantages of the lumen geometries described herein—particularly increased cross-sectional flow area for a given outer diameter—may be most pronounced for compressible gases, which can exhibit greater pneumatic compliance and flow resistance effects that limit frequency response. Accordingly, the disclosed lumen geometries and pneumatic pathways can be implemented with air or other gases (and, in some embodiments, liquids) to achieve suitable pressure sensing performance for the intended clinical application.

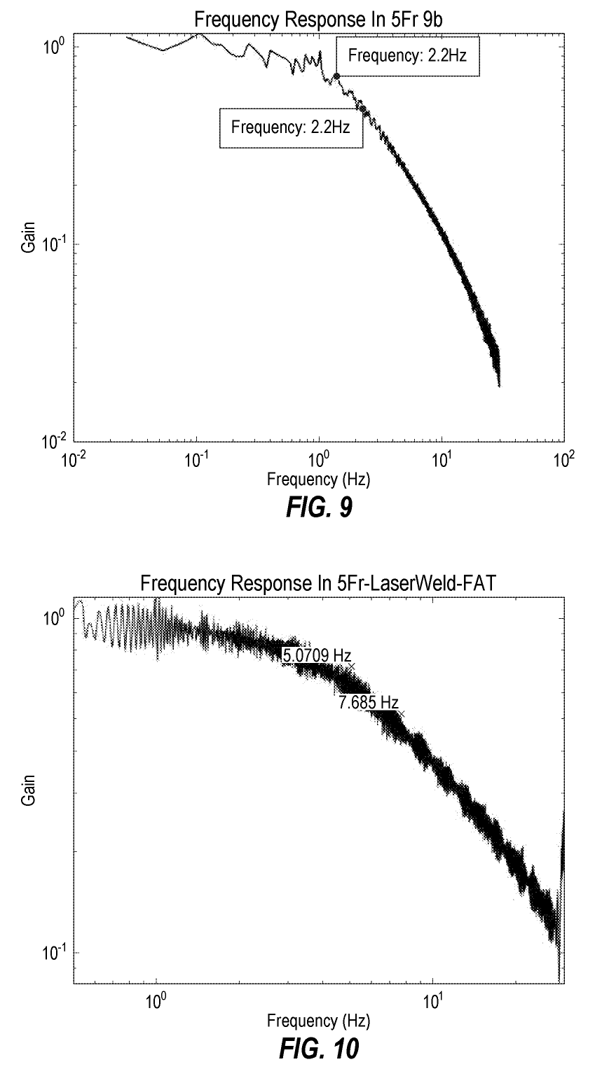

The increased cross-sectional flow area of a non-circular secondary lumen (for a given catheter outer diameter) can reduce pneumatic damping of a pressure signal transmitted through the secondary lumen, thereby improving frequency response relative to a circular secondary lumen constrained within the same outer diameter. FIG. 9 illustrates an example frequency-response characterization for a conventional pressure-sensing catheter having secondary lumens with a circular cross-sectional shape, and FIG. 10 illustrates an example frequency-response characterization for a pressure-sensing catheter having secondary lumens with a non-circular cross-sectional shape in accordance with embodiments of the present disclosure. In the illustrated examples, the plots show the amplitude of the pressure signal measured at a proximal sensing location (e.g., at a transducer coupled to the secondary lumen) relative to the amplitude of an applied or reference pressure signal, as a function of frequency.

In FIGS. 9 and 10, reference levels (e.g., about 70% and about 50% amplitude) are shown to illustrate attenuation as frequency increases. As depicted, both catheters measure approximately the full amplitude of the pressure signal at relatively low frequencies. As the frequency increases, the catheter of FIG. 9 exhibits increased attenuation (e.g., a reduction in normalized amplitude), indicating decreased ability to transmit higher-frequency components of the pressure signal through the secondary lumen. By contrast, the catheter of FIG. 10 maintains a higher normalized amplitude at higher frequencies, reflecting improved pressure-signal transmission. For example, at 10Hz in the illustrated data, the catheter of FIG. 10 exhibits a higher measured amplitude than the catheter of FIG. 9.

In one tested example, the one or more secondary lumens 110 of the catheter used to acquire the measurements in FIG. 10 exhibit a cross-sectional area of about 2.86 times the cross-sectional area of the secondary lumen(s) of the catheter used to acquire the measurements in FIG. 9. This increase in secondary-lumen flow area can improve frequency response by reducing damping and flow resistance in the pneumatic pathway, thereby enhancing sensing capability relative to conventional pressure-sensing catheters having circular secondary lumens. Additionally, due to the larger secondary-lumen cross-sectional area, the one or more secondary lumens 110 may better tolerate manufacturing variation, including shrinkage and/or partial occlusion that can occur during thermal processing and distal assembly, thereby reducing susceptibility to sensing failure.

It is believed that the pressure-sensing catheters, according to embodiments of the present disclosure, owing to their specific ratio of cross-sectional area of the secondary lumens and the primary lumen to a total cross-sectional area of the pressure-sensing catheter and the specific non-circular shape of the secondary lumens, are capable of translating changes in pressure in a body cavity to pressure signals with higher amplitudes in comparison with conventional pressure-sensing catheters (e.g., catheters with circular cross-sectional area). This allows detection of pressure change in a cavity with higher accuracy. Moreover, the specific ratio of cross-sectional area of the secondary lumens and the primary lumen to a total cross-sectional area of the pressure-sensing catheter allows higher quality of manufacturing without occlusion of the secondary lumens taking place.

Additional non-limiting example embodiments of the present disclosure are set forth below.

Embodiment 1. A pressure-sensing catheter, comprising: an elongate catheter body extending from a proximal end to a distal end and having an outer surface; a primary lumen extending along at least a portion of the elongate catheter body; one or more secondary lumens extending along at least a portion of the elongate catheter body, each secondary lumen having a non-circular cross-sectional shape; and at least one collapsible vessel disposed on the outer surface of the elongate catheter body, the collapsible vessel being in fluid communication with a secondary lumen of the one or more secondary lumens.

Embodiment 2. The pressure-sensing catheter of Embodiment 1, wherein the catheter body has an outer diameter of about 2.0mm or less.

Embodiment 3. The pressure-sensing catheter of Embodiment 2, wherein the outer diameter is about 1.67mm or less.

Embodiment 4. The pressure-sensing catheter of any of Embodiments 1-3, wherein the non-circular cross-sectional shape is elliptical or D-shaped.

Embodiment 5. The pressure-sensing catheter of any of Embodiments 1-4, wherein a minimum distance between at least one of the one or more secondary lumens and the outer surface of the catheter body is between about 0.15mm and about 0.25mm.

Embodiment 6. The pressure-sensing catheter of any of Embodiments 1-5, comprising a plurality of collapsible vessels disposed at different circumferential positions around the catheter body, each collapsible vessel being in fluid communication with a respective secondary lumen.

Embodiment 7. The pressure-sensing catheter of any of Embodiments 1-6, wherein the non-circular cross-sectional shape is an elliptical shape having a length along a major axis in a range extending from about 0.20mm to about 0.55mm, and a width along a minor axis in a range extending from about 0.15mm to about 0.25mm.

Embodiment 8. The pressure-sensing catheter of any of Embodiments 1-7, wherein the primary lumen has a D-shaped cross-sectional area.

Embodiment 9. The pressure-sensing catheter of any of Embodiments 1-8, wherein the primary lumen has a generally rectangular cross-sectional shape with inwardly arcing sides.

Embodiment 10. The pressure-sensing catheter of any of Embodiments 1-9, wherein the one or more secondary lumens comprise two secondary lumens located on opposite sides of the primary lumen.

Embodiment 11. The pressure-sensing catheter of Embodiment 10, wherein the two secondary lumens have a same cross-sectional size and shape.

Embodiment 12. The pressure-sensing catheter of any of Embodiments 1-11, wherein the one or more secondary lumens have a cross-sectional area of 0.050 square mm or less.

Embodiment 13. The pressure-sensing catheter of Embodiment 12, wherein the one or more secondary lumens have a cross-sectional area of 0.025 square mm or less.

Embodiment 14. The pressure-sensing catheter of any of Embodiments 1-13, wherein a ratio of a cross-sectional area of the primary lumen and the one or more secondary lumens to a total cross-sectional area of the catheter body is 0.25 or more.

Embodiment 15. A pressure-sensing medical device, comprising: a pressure-sensing catheter including an elongate catheter body extending from a proximal end to a distal end and having an outer surface, a primary lumen extending along at least a portion of the elongate catheter body, one or more secondary lumens extending along at least a portion of the elongate catheter body, the one or more secondary lumens exhibiting a non-circular cross-sectional shape, and at least one collapsible vessel disposed on the body, each collapsible vessel in fluid communication with a secondary lumen of the one or more secondary lumens; and a pressure-sensing transducer located and configured to sense a pressure within the at least one collapsible vessel and the secondary lumen of the one or more secondary lumens.

Embodiment 16. The pressure-sensing medical device of Embodiment 15, wherein the non-circular cross-sectional shape is an elliptical shape having a length along a major axis in a range extending from about 0.20mm to about 0.55mm, and a width along a minor axis in a range extending from about 0.15mm to about 0.25mm.

Embodiment 17. The pressure-sensing medical device of Embodiment 15 or 16, wherein the one or more secondary lumens have a cross-sectional area of 0.050 square mm or less.

Embodiment 18. A method of manufacturing a pressure-sensing catheter, the method comprising: extruding an elongate catheter shaft having a proximal end and a distal end, the catheter shaft including a polymeric shaft wall defining a primary lumen extending between the proximal and distal ends and at least one secondary lumen extending between the proximal and distal ends, wherein the at least one secondary lumen has a non-circular cross-sectional shape; forming, in a distal portion of the catheter shaft, at least one opening extending between the at least one secondary lumen and an exterior of the catheter shaft; positioning a compliant member about the distal portion of the catheter shaft over the at least one opening, the compliant member having a proximal cuff portion and a distal cuff portion contacting an outer surface of the catheter shaft; and laser-welding the compliant member to the catheter shaft to form a sealed chamber in fluid communication with the at least one secondary lumen. Laser-welding comprises performing a first low-power laser pass along a first side of the compliant member, the first low-power laser pass beginning adjacent a first edge of the compliant member and translating axially away from the compliant member to apply heat sufficient to conform the compliant member to the catheter shaft, performing a second low-power laser pass along the first side, the second low-power laser pass beginning adjacent the first edge of the compliant member and translating axially away from the compliant member to melt and fuse compliant member material to shaft material to create a first weld seam, performing a third low-power laser pass along a second side of the compliant member opposite the first side, the third low-power laser pass beginning adjacent a second edge of the compliant member and translating axially away from the compliant member, and performing a fourth low-power laser pass along the second side, the fourth low-power laser pass beginning adjacent the second edge of the compliant member and translating axially away from the compliant member to melt and fuse compliant member material to shaft material to create a second weld seam, wherein the first and second weld seams cooperate to seal the compliant member to the catheter shaft while maintaining patency of the primary lumen and the at least one secondary lumen.

Embodiment 19. The method of Embodiment 18, further comprising wetting the compliant member with alcohol to facilitate positioning prior to laser-welding, and evaporating residual alcohol during the first low-power laser pass.

Embodiment 20. The method of Embodiment 18 or 19, further comprising inserting a temporary mandrel into at least one of the primary lumen or the at least one secondary lumen during laser-welding and removing the mandrel after laser-welding.

The embodiments of the disclosure described above and illustrated in the accompanying drawings do not limit the scope of the disclosure, which is encompassed by the scope of the appended claims and their legal equivalents. Any equivalent embodiments are within the scope of this disclosure. Indeed, various modifications of the disclosure, in addition to those shown and described herein, such as alternate useful combinations of the elements described, will become apparent to those skilled in the art from the description. Such modifications and embodiments also fall within the scope of the appended claims and equivalents.

What is claimed is:

1. A pressure-sensing catheter, comprising:
an elongate catheter body extending from a proximal end to a distal end and having an outer surface;
a primary lumen extending along at least a portion of the elongate catheter body;
one or more secondary lumens extending along at least a portion of the elongate catheter body, each secondary lumen having a non-circular cross-sectional shape; and
at least one collapsible vessel disposed on the outer surface of the elongate catheter body, the collapsible vessel being in fluid communication with a secondary lumen of the one or more secondary lumens.

2. The pressure-sensing catheter of claim 1, wherein the catheter body has an outer diameter of about 2.0mm or less.

3. The pressure-sensing catheter of claim 2, wherein the outer diameter is about 1.67mm or less.

4. The pressure-sensing catheter of claim 1, wherein the non-circular cross-sectional shape is elliptical or D-shaped.

5. The pressure-sensing catheter of claim 1, wherein a minimum distance between at least one of the one or more secondary lumens and the outer surface of the catheter body is between about 0.15mm and about 0.25mm.

6. The pressure-sensing catheter of claim 1, comprising a plurality of collapsible vessels disposed at different circumferential positions around the catheter body, each collapsible vessel being in fluid communication with a respective secondary lumen.

7. The pressure-sensing catheter of claim 1, wherein the non-circular cross-sectional shape is an elliptical shape having a length along a major axis in a range extending from about 0.20mm to about 0.55mm, and a width along a minor axis in a range extending from about 0.15mm to about 0.25mm.

8. The pressure-sensing catheter of claim 1, wherein the primary lumen has a D-shaped cross-sectional area.

9. The pressure-sensing catheter of claim 1, wherein the primary lumen has a generally rectangular cross-sectional shape with inwardly arcing sides.

10. The pressure-sensing catheter of claim 1, wherein the one or more secondary lumens comprise two secondary lumens located on opposite sides of the primary lumen.

11. The pressure-sensing catheter of claim 10, wherein the two secondary lumens have a same cross-sectional size and shape.

12. The pressure-sensing catheter of claim 1, wherein the one or more secondary lumens have a cross-sectional area of 0.050 square mm or less.

13. The pressure-sensing catheter of claim 12, wherein the one or more secondary lumens have a cross-sectional area of 0.025 square mm or less.

14. The pressure-sensing catheter of claim 1, wherein a ratio of a cross-sectional area of the primary lumen and the one or more secondary lumens to a total cross-sectional area of the catheter body is 0.25 or more.

15. A pressure-sensing medical device, comprising:
a pressure-sensing catheter including:
an elongate catheter body extending from a proximal end to a distal end and having an outer surface;
a primary lumen extending along at least a portion of the elongate catheter body;
one or more secondary lumens extending along at least a portion of the elongate catheter body, the one or more secondary lumens exhibiting a non-circular cross-sectional shape; and
at least one collapsible vessel disposed on the body, each collapsible vessel in fluid communication with a secondary lumen of the one or more secondary lumens; and
a pressure-sensing transducer located and configured to sense a pressure within the at least one collapsible vessel and the secondary lumen of the one or more secondary lumens.

16. The pressure-sensing medical device of claim 15, wherein the non-circular cross-sectional shape is an elliptical shape having a length along a major axis in a range extending from about 0.20mm to about 0.55mm, and a width along a minor axis in a range extending from about 0.15mm to about 0.25mm.

17. The pressure-sensing medical device of claim 15, wherein the one or more secondary lumens have a cross-sectional area of 0.050 square mm or less.

18. A method of manufacturing a pressure-sensing catheter, the method comprising:
extruding an elongate catheter shaft having a proximal end and a distal end, the catheter shaft including a polymeric shaft wall defining a primary lumen extending between the proximal and distal ends and at least one secondary lumen extending between the proximal and distal ends, wherein the at least one secondary lumen has a non-circular cross-sectional shape;
forming, in a distal portion of the catheter shaft, at least one opening extending between the at least one secondary lumen and an exterior of the catheter shaft;
positioning a compliant member about the distal portion of the catheter shaft over the at least one opening, the compliant member having a proximal cuff portion and a distal cuff portion contacting an outer surface of the catheter shaft; and
laser-welding the compliant member to the catheter shaft to form a sealed chamber in fluid communication with the at least one secondary lumen, wherein laser-welding comprises:
performing a first low-power laser pass along a first side of the compliant member, the first low-power laser pass beginning adjacent a first edge of the compliant member and translating axially away from the compliant member to apply heat sufficient to conform the compliant member to the catheter shaft;
performing a second low-power laser pass along the first side, the second low-power laser pass beginning adjacent the first edge of the compliant member and translating axially away from the compliant member to melt and fuse compliant member material to shaft material to create a first weld seam;
performing a third low-power laser pass along a second side of the compliant member opposite the first side, the third low-power laser pass beginning adjacent a second edge of the compliant member and translating axially away from the compliant member; and
performing a fourth low-power laser pass along the second side, the fourth low-power laser pass beginning adjacent the second edge of the compliant member and translating axially away from the compliant member to melt and fuse compliant member material to shaft material to create a second weld seam;
wherein the first and second weld seams cooperate to seal the compliant member to the catheter shaft while maintaining patency of the primary lumen and the at least one secondary lumen.

19. The method of claim 18, further comprising wetting the compliant member with alcohol to facilitate positioning prior to laser-welding, and evaporating residual alcohol during the first low-power laser pass.

20. The method of claim 18, further comprising inserting a temporary mandrel into at least one of the primary lumen or the at least one secondary lumen during laser-welding and removing the mandrel after laser-welding.
